(19) United States
(12) Patent Application Publication
(10) Pub. No.: US 2025/0235157 A1
(43) Pub. Date: Jul. 24, 2025

(54) HIGHLY BREATHABLE ELECTRONIC PATCH AND METHOD FOR PRODUCING SUCH PATCH

(71) Applicant: Covestro Deutschland AG, Leverkusen (DE)

(72) Inventors: Gerd Bueschel, Admannshagen-Bargeshagen (DE); Klas-Moritz Kossel, Dormagen (DE)

(21) Appl. No.: 18/848,976

(22) PCT Filed: Apr. 12, 2023
(86) PCT No.: PCT/EP2023/059545

§ 371 Date: Sep. 20, 2024

(30) Foreign Application Priority Data
Apr. 13, 2022 (EP) 22168147.1

(51) Int. Cl. A61B 5/00; A61B 5/01

(52) U.S. Cl. CPC A61B 5/6833; A61B 5/01; A61B 5/443; A61B 2562/12; A61B 2562/18

(57) ABSTRACT

The invention is related to an electronic patch (EP) comprising at least:
(A) a first polymeric layer (A) with a first surface a) and a second surface b) which is parallel to surface a), whereby layer (A) provides a water vapour permeability of ≥500 g/m²d;
(B) an electronic device (B) providing at least one flat surface which is positioned on the first surface a) of the first polymeric layer (A);
(C) optionally a second polymeric layer (C) providing a vapour permeability of ≥500 g/m²d, preferably in a range of 500 to 2000 g/m²d;
(D) optionally a further layer (D) as water barrier positioned between the electronic device (B) and the first polymeric layer (A);
(E) optionally an adhesive on the second surface b) of the first polymeric layer (A);
(F) optionally a polymeric foam (F) with a moisture vapour transmission rate (MVTR) of ≥500 g/m²d, more preferably in a range of 550 to 2000 g/m²d, most preferably in a range of 600 to 1500 g/m²d covering the electronic device (B), determined according to DIN 53122-1:2001-08;
   whereby the electronic patch (EP) is surrounded by the first polymeric layer (A) and optionally the second polymeric layer (C) in a way that the overall water vapour permeability of the electronic patch (EP) is ≥400 g/m²d as well as a process for the production of the electronic patch (EP).

The invention is related to an electronic patch (EP) providing at least one flat surface ep), the (EP) comprising at least one first polymeric layer (A) and an electronic device (B), optionally a second polymeric layer (C), a further layer (D) and a foam (F) with specific properties, and also optionally an adhesive (E), whereby ≥50%, preferably ≥60%, more preferably ≥70%, more preferably ≥80% of the total surface of the flat surface ep) of the electronic patch (EP) is superimposed by material providing an overall moisture vapour transmission rate (MVTR) of ≥400 g/m²d. Furthermore, the invention is related to a process for producing an electronic patch (EP) according to the invention as well as its use for the application on the skin of a user.

There exist a lot of electronic patches on the market for the detection of life parameters like temperature, electrolytes and other parameters detectable on the skin of a person which should be usable for more than one day. However, most of these patches show less breathability which causes skin irritations on the skin of the user. This is because the electronic patch needs to be protected against water as the user should not be hindered in daily life by putting on and taking off the patch for each shower.

Therefore, it exists a need to develop an electronic patch that incorporates safeness, accuracy in combination with wearing comfort and skin friendliness which can be used in daily life of a user.

A first aspect of the invention is related to an electronic patch (EP) providing at least one flat surface ep) which is arranged to be placed on the skin of a user, the electronic patch (EP) comprising at least:
(A) a first polymeric layer (A) with a first surface a) and a second surface b) which is parallel to surface a), whereby layer (A) provides a moisture vapour transmission rate (MVTR) of ≥500 g/m²d, determined according to DIN 53122-1:2001-08;
(B) an electronic device (B) providing at least one flat surface b) which is positioned on the first surface a) of the first polymeric layer (A), wherein the electronic device (B) is optionally surrounded by a water barrier (WB);
(C) optionally a second polymeric layer (C) providing a moisture vapour transmission rate (MVTR) of ≥500 g/m²d, preferably in a range of 500 to 2000 g/m²d, more preferably in a range of 600 to 1500 g/m²d determined according to DIN 53122-1:2001-08;
(D) optionally at least one further layer (D) as water barrier (WB) positioned between the electronic device (B) and the first polymeric layer (A) or between the electronic device (B) and the second polymeric layer (C) or both;
(E) optionally an adhesive (E) on the second surface b) of the first polymeric layer (A);
(F) optionally a polymeric foam (F) with a moisture vapour transmission rate (MVTR) of ≥500 g/m²d, more preferably in a range of 550 to 2000 g/m²d, most preferably in a range of 600 to 1500 g/m²d covering the electronic device (B), determined according to DIN 53122-1:2001-08;
   whereby a surface area ep1) with a dimension of ≥50%, preferably ≥60%, more preferably ≥70%, even more preferably ≥80% of the total surface of the flat surface ep) of the electronic patch (EP) is superimposed by material providing an overall moisture vapour transmission rate (MVTR) of ≥400 g/m²d, determined according to DIN 53122-1:2001-08.

If the first polymeric layer (A) forms the surface of the electronic patch (EP), which is arranged to come into contact with the user, the surfaces, surface ep) and surface b) are identical. As surface a) of the first polymeric layer (A) is parallel to surface b) the dimensions of the surface a) and surface ep) are also the identical.

The user may be any living organism, preferably a human being or an animal.

The surface ep) of the electronic patch (EP) is defined as the surface which superimposes the skin of a user when the electronic patch (EP) is installed to be used for example for the monitoring of a function of the skin or the body of the user. Preferably, the surface (ep) is formed at least partially by the first polymeric layer (A), preferably by surface b) of polymeric layer (A).

The polymer of the first polymeric layer (A) preferably has a thickness in a range of from 10 to 1000 μm. The polymer selected to build polymer layer (A) can be selected of any polymer that provides a moisture vapour transmission rate (MVTR) of ≥500 g/m²d in the thickness range of 10 to 1000 μm. The first surface a) of the first polymeric layer (A) comprises a first surface area ep1) and a second surface area ep2). The first surface area ep1) preferably covers ≥30%, preferably ≥ 50%, more preferably ≥70%, even more preferably ≥80% of the total surface of the flat surface a). Preferably, ≥30%, more preferably ≥50%, even more preferably ≥70%, most preferably ≥80% of the surface a) or surface ep) of the electronic patch (EP) is formed by material providing an overall moisture vapour transmission rate (MVTR) of ≥400 g/m²d, preferably of ≥500 g/m²d.

Preferably, the dimension of the total surface ep) is in a range of from 0.5 cm² to 10 m², more preferably in a range of from 1 cm² to 1 m², even more preferably in a range of from 3 cm² to 50 cm².

On the surface ep) the electronic device (B) is positioned, preferably in a second surface area ep2) of the first polymeric layer (A). The electronic device (B) preferably covers or superimposes an area of ≤70%, preferably of ≤50%, more preferably of ≤30%, most preferably ≤20% of the surface ep), based on the total dimension of the total surface ep). The electronic device (B) preferably covers or superimposes an area of ≤70%, preferably of ≤50%, more preferably of ≤30%, most preferably ≤20% of the surface a), based on the total dimension of the total surface a).

Preferably, the volume of the electronic device (B) is in a range of from 0.01 cm³ to 1 m³, more preferably in a range of from 0.02 cm³ to 50 cm³, even more preferably in a range of from 0.05 cm³ to 10 cm³. Preferably, the electronic device (B) is surrounded by the further layer (D).

Preferably, the length or width or height of the electronic device (B) independently is in a range of from 0.1 cm to 1 m, more preferably in a range of from 1 cm to 50 cm, even more preferably in a range of from 2 cm to 10 cm.

The optional second polymeric layer (C) preferably has a thickness in a range of from 10 to 1000 μm. The polymer selected to build polymeric layer (C) can be selected of any polymer or polymer mixture that provides a moisture vapour transmission rate (MVTR) of ≥500 g/m²d in the thickness range of 10 to 1000 μm. Preferably, the polymeric of layer (C) is the same as the polymer of layer (A). The second polymeric layer (C) is preferably positioned on top of the further layer (D) which is positioned on the electronic device (B). Especially, if the first polymeric layer (A) solely builds the base of the electronic patch (EP) the second polymeric layer (C)

The optional further layer (D) can be any layer that provides a water barrier function. Preferably, the material of the further layer (D) is selected of the group consisting of a metal like aluminum, a polymer foil, a foam, a foil/foam combination, a coated polymeric foil, a glass, a ceramic or a combination of at least two thereof. Preferably, the material of the further layer (D) is a polymer either in form of a foil, a foam or a foil/foam combination. The further layer (D) preferably is produced by printing, injection molding, casting, extrusion or a combination of these methods. In case the further layer (D) is present, the further layer (D) is positioned on top of the first polymeric layer (A) underneath the electronic device (B) and on top of the electronic device (B) in a way that the electronic device (B) preferably is surrounded by the further layer (D).

The optional further layer (D) may be provided between the first polymeric layer (A) and the electronic device (B) by any means the person skilled in the art would select. Preferably, the providing of the further layer (D) is selected from the group consisting of screen printing, brushing, submitting a film or a combination of at least two thereof. Preferably, the providing in step (II) is performed by a screen printing process of a material with an MVTR of ≤500 g/m²d. If not provided directly onto the surface of the electronic device (B), the further layer (D) is preferably printed directly onto the first polymeric layer (A) and optionally second polymeric layer (C).

The further layer (D) may be printed within the electronic printing process where electronics, symbols, holograms or the like are printed to form the electronic device (B). The further layer (D) preferably has a thickness in a range of from 10 to 1000 μm. The polymer selected to build polymeric layer (D) can be selected of any polymer that provides a moisture vapour transmission rate (MVTR) of ≤500 g/m²d in the thickness range of 10 to 1000 μm.

The optional adhesive (E) which is placed on the second surface b) of the first polymeric layer (A) can be any adhesive that the person skilled in the art would select for contacting the skin of a user. Preferably, the adhesive (E) is selected from the group consisting of a polyurethane adhesive, an acrylic adhesive, silicone adhesive, hydrogel adhesive or a combination or mixture of at least two thereof.

The optional polymeric foam (F) is at least partially surrounded by any of the other layers, like the first layer (A) or the optional layer (C) or both. Preferably, the foam (F) has no contact to the surrounding. The material of the polymeric foam (F) is preferably selected from the group consisting of polyurethane, silicone, polyolefinic polymers like polyethylene, polypropylene or the like, or a combination or mixture of at least two thereof. The polymeric foam (F) is preferably positioned between the first polymeric layer (A) and/or the second polymeric layer (C) and the further layer (D). Alternatively, or in addition the foam can be positioned between the further layer (D) and the electronic device (B). Preferably, the foam is applied between all polymeric layers (A), (C) and the further layer (D).

The first surface area ep1) builds a part of the surface ep) and also of the surface a), if the first polymeric layer (A) also builds the surface ep). The surface area ep1) covers a dimension of ≥30%, preferably of ≥50%, more preferably of ≥70%, even more preferably of ≥80% of the total surface of the flat surface ep) or a) of the electronic patch (EP). The surface area ep1) is superimposed by material providing an overall moisture vapour transmission rate (MVTR) of ≥400 g/m²d, preferably of ≥500 g/m²d, determined according to DIN 53122-1:2001-08. Preferably, the surface area ep1) is only partially superimposed by the electronic device (B) or the further layer (D). More preferably, the surface area ep1) is not superimposed by the electronic device (B) or the further layer (D). The electronic device (B) and the further layer (D) are preferably positioned on the second surface area ep2) which is also part of the surface ep) or a).

In a preferred embodiment of the electronic patch (EP) ≥50%, more preferably ≥60%, most preferably ≥70%, of the total volume of the electronic patch (EP) is filled with a material that provides a moisture vapour transmission rate (MVTR) greater than 500 g/m²d, determined according to DIN 53122-1:2001-08.

The total volume of the electronic patch (EP) is the volume that is surrounded by the outer layers of the electronic patch (EP). The total volume of the electronic patch (EP) may incorporate different areas. One area is filled by the electronic device (B). Preferably, the electronic device (B) is surrounded by a water barrier (WB). This is the reason why the moisture vapour transmission rate (MVTR) of this area is ≤500 g/m²d. This area preferably fills out ≤50%, more preferably ≤40%, even more preferably ≤30%, most preferably ≤20%, extremely preferably ≤10%, of the total volume of the electronic patch (EP).

Preferably, the material which fills out the area with a moisture vapour transmission rate (MVTR) of ≥500 g/m²d is selected from air, a foam or any polymeric material with an MVTR of ≥500 g/m²d.

In a preferred embodiment of the electronic patch (EP) the polymer of the first polymeric layer (A) and optionally the second polymeric layer (C) is selected from the group consisting of a thermoplastic elastomer (e.g. thermoplastic polyurethane), a silicone, a (expanded) polytetrafluorethylene (PTFE), a polyamide (e.g. a polyphthalamide), a polyblockamide, a polyacrylate, a polycarbonate, a co-polycarbonate, a polyether-blockamide (e.g. Platilon® M2234), a polyester, a co-polyester, a polyimide, a polyvinylchloride, a polyolefine or a combination of at least two thereof. Preferably, the polymeric layer (A) comprises the polymer in an amount in a range of from 60 to 100 wt.-%, more preferably of from 80 to 99 wt.-%, even more preferably of from 90 to 97 wt.-%, based on the total weight of the polymeric layer (A). The polymeric layer (A) preferably comprise additives like UV stabilizers, plasticizers, deformers, pigments and any other organic or inorganic components in a total amount of from 1 to 20 wt.-%, preferably of from 2 to 15 wt.-%, even more preferably of from 5 to 10 wt.-%.

In a preferred embodiment of the electronic patch (EP) the first polymeric layer (A) and optionally the second polymeric layer (C) provides at least one, preferably at least two, more preferably all of the following properties:
(A1) a thickness in a range of 10 to 500 μm, more preferably in a range of 20 to 400 μm, most preferably in a range of 25 to 120 μm;
(A2) a surface area in a range of 1 to 1000 cm², more preferably in a range of 10 to 800 cm², most preferably in a range of 50 to 500 cm²;
(A3) an aspect ratio of thickness to length or width building the surface area, preferably the aspect ratio of thickness to length, is in a range of 1:10 to 1:10000, more preferably in a range of 1:20 to 1:5000, most preferably in a range of 1:100 to 1:1000;
(A4) a vapour permeability of ≥500 g/m²d, more preferably of ≥600 g/m²d, most preferably of ≥700 g/m²d;
(A5) a vapour permeability in a range of 500 to 3000 g/m²d, more preferably in a range of 550 to 2500 g/m²d, most preferably in a range of 600 to 1600 g/m²d.

Preferably, the first polymeric layer (A) provides one property or one property combination selected from the group consisting of (A1); (A2); (A3); (A4); (A5); (A1) and (A2); (A1) and (A3); (A1) and (A4); (A1) and (A5); (A2) and (A3); (A2) and (A4); (A2) and (A5); (A3) and (A4); (A3) and (A5); (A4) and (A5); (A1) and (A2) and (A3); (A1) and (A2) and (A4); (A1) and (A2) and (A5); (A1) and (A3) and (A4); (A1) and (A3) and (A5); (A1) and (A4) and (A5); (A2) and (A3) and (A4); (A2) and (A3) and (A5); (A2) and (A4) and (A5); (A3) and (A4) and (A5); (A1) and (A2) and (A3) and (A4); (A1) and (A2) and (A3) and (A5); (A1) and (A2) and (A4) and (A5); (A1) and (A3) and (A4) and (A5); (A2) and (A3) and (A4) and (A5); (A1) and (A2) and (A3) and (A4) and (A5).

Preferably, the vapour permeability of the first polymeric layer (A) and optionally of the second polymeric layer (C) lies in a range of ≥500 to 3000 g/m²d, more preferably in a range of ≥600 to 2500 g/m²d, most preferably in a range of ≥700 to 1600 g/m²d.

In a preferred embodiment of the electronic patch (EP) the electronic device (B) provides at least one, preferably at least two, more preferably all of the following properties:
(B1) a thickness in a range of 0.5 μm to 5 cm, more preferably in a range of 10 μm to 1 cm, most preferably in a range of 2 μm to 5 mm;
(B2) a volume in a range of 0.5 to 500 mm³, more preferably in a range of 5 to 400 mm³, most preferably in a range of 10 to 250 mm³;
(B3) optionally, the electronic device (B) is surrounded by a water barrier (WB);
(B4) at least one electronic component selected from the group consisting of antenna, battery, wires, rigid PCB boards, flexible PCB boards, chips, memory elements, sensors or combinations of at least two thereof;
Preferably, the electronic device (B) provides one property or one property combination selected from the group consisting of (B1); (B2); (B3); (B4); (B1) and (B2); (B1) and (B3); (B1) and (B4); (B2) and (B3); (B2) and (B4); (B3) and (B4); (B1) and (B2) and (B3); (B1) and (B2) and (B4); (B1) and (B3) and (B4); (B2) and (B3) and (B4); (B1) and (B2) and (B3) and (B4).

In a preferred embodiment of the electronic patch (EP), at least one of the at least one further layer (D) is present and is positioned between the electronic device (B) and the first polymeric layer (A), and optionally between the electronic device (B) and the second polymeric layer (C); preferably the further layer (D) is positioned only in the area where the electronic device (B) is positioned on the first polymeric layer (A) or the second polymeric layer (C).

Preferably, at least one of the further layers (D) has the function as water barrier to protect the electronic device (B) from water vapour and water. The at least one further layer (D) therefore surrounds the electronic device (B) preferably totally.

In a preferred embodiment of the electronic patch the ratio of the surface area of the first polymeric layer (A) to the surface area of the first layer (D) is in a range of from 100:1 to 1.5:1, preferably in a range of from 90:1 to 5:1, most preferable in a range of from 50:1 to 20:1. Especially, the ratio of the surface area ep1) of the first polymeric layer (A) to the surface area of the first layer (D) is in a range of from 100:1 to 1.5:1, preferably in a range of from 90:1 to 5:1, most preferable in a range of from 50:1 to 20:1.

In a preferred embodiment of the electronic patch (EP) the further layer (D) is present and provides at least one, preferably at least two, more preferably all of the following properties:
(D1) a thickness in a range of 1 to 500 μm, more preferably in a range of 15 to 200 μm, most preferably in a range of 40 to 150 μm;
(D2) a surface area in a range of 0.1 to 900 cm², more preferably in a range of 1 to 800 cm², most preferably in a range of 10 to 500 cm²;
(D3) a vapour permeability smaller than 400 g/m²d, more preferably smaller than 200 g/m²d, most preferably smaller than 100 g/m²d.

Preferably, the first further layer (D) provides one property or one property combination selected from the group consisting of (D1); (D2); (D3); (D1) and (D2); (D1) and (D3); (D2) and (D3); (D1) and (D2) and (D3).

In a preferred embodiment of the electronic patch (EP), at least one of the at least one further layer (D) is present and is made of a metal like aluminum, a polymer foil, a foam, a foil/foam combination, a coated polymeric foil, a glass, a ceramic or a combination of at least two thereof.

In a preferred embodiment of the electronic patch (EP), at least one of the at least one further layer (D) is present and is made of a polymer, preferably a polymer foil or foam or foil/foam combination, selected of the group consisting of polyethylene, polypropylene, polyethylene terephthalate, polystyrene, polyvinylchloride, polyamide, preferably polyethylene terephthalate or a mixture of at least two thereof. Preferably, the further layer (D) comprises the polymer in an amount in a range of from 60 to 100 wt.-%, more preferably of from 80 to 99 wt.-%, even more preferably of from 90 to 97 wt.-%, based on the total weight of the further layer (D).

In a preferred embodiment of the electronic patch (EP) the polymeric foam (F) is present and is selected from the group consisting of polyurethanes, thermoplastic polyurethanes, polyolefinic material, silicones or a mixture or combination of at least two thereof. Preferably, the polymeric foam (F) is made of a thermoplastic polyurethane.

A further aspect of the invention refers to a process for producing an electronic patch (EP), providing a flat surface ep) on its outside, the process comprising at least the following steps:
(I) providing a flat first part of a first polymeric layer (A) with a first surface a) comprising a first surface area ep1) and a second surface area ep2), wherein the first polymeric layer (A) provides a vapour permeability in a range of 500 to 3000 g/m²d, more preferably in a range of 550 to 2500 g/m²d, most preferably in a range of 600 to 1600 g/m²d;
(II) optionally, providing a further layer (D) on the second surface area ep2) of the first part of the first polymeric layer (A) to cover ≤70%, preferably ≤50%, more preferably ≤ 30%, most preferably ≤20% of the first part of the first polymeric layer (A);
(III) optionally applying of at least one electrical pathway onto the first polymeric layer (A) and/or the further layer (D; preferably onto the further layer (D);
(IV) providing an electronic device (B) on the first part of the first polymeric layer (A) in a way that the electronic device (B) superimposes the polymeric layer (A) in the second surface area ep2) of the polymer layer (A). The electronic device (B) is preferably separated from the polymeric layer (A) by providing a further layer (D) as water barrier between the electronic device (B) and the first part of the first polymeric layer (A). Preferably, the electronic devise (B) is positioned on the further layer (D). Alternatively or additionally the electronic device (B) itself may be waterproof;
(V) optionally, providing a second further layer (D) on the electronic device (B) and if present also onto the electrical pathways;
(VI) optionally providing a polymeric foam (F) with a vapour permeability in a range of 500 to 1000 g/m²d, more preferably in a range of 550 to 950 g/m²d, most preferably in a range of 600 to 900 g/m²d covering the electronic device (B);
(VII) covering the electronic device (B) by a second part of the first polymeric layer (A) or a second polymeric layer (C) in a way that overlapping edges of the first part of the first polymeric layer (A) and the second part of the first polymeric layer (A) or of the first part of the first polymeric layer (A) and the second polymeric layer (C) are formed;
(VIII) fixing the overlapping edges of the first part of the first polymeric layer (A) and the second part of the first polymeric layer (A) or fixing the second polymeric layer (C) with the first part of the first polymeric layer (A) achieving an electronic patch (EP) which is totally surrounded by the first polymeric layer (A) or partially surrounded by the first polymeric layer (A) and partially surrounded by the second polymeric layer (C) and the electronic device (B) is surrounded by the polymeric layers (A) and optionally (C);
(IX) optionally providing an adhesive onto the underside of the electronic patch (EP);
   whereby the first surface area ep1) which covers ≥50%, preferably ≥60%, more preferably ≥70%, even more preferably ≥80% of the total surface of the flat surface ep) or surface a) of the electronic patch (EP) is superimposed by material providing an overall moisture vapour transmission rate (MVTR) of ≥400 g/m²d.

Preferably, the sequence of the steps (I) to (VIII) is as listed above.

To measure the surface of the electronic patch (EP) which is superimposed by material providing an overall moisture vapour transmission rate (MVTR) of ≥400 g/m²d all material is taken into account that propagates in an angle of 90°±5° from the surface of the first polymeric layer (A) in the situation when the first polymeric layer (A) is positioned on a flat support like a table.

It is preferred that the dimensions and orientation of the first polymeric layer (A), optionally second polymeric layer (C) and optionally further layer (D) are chosen in a way that the overall breathability of the electronic patch (EP) is in a range of 50 to 100% of the breathability of the first polymeric layer (A).

Providing of polymeric layer (A) in step (I) can be established by any means the person skilled in the art would select for the process. The providing in step (I) is preferably selected from the group consisting of a roll to roll process, on a belt, on a conveyer, on a substrate or a combination thereof.

Optionally providing a further layer (D) on the second surface area ep2) of the first part of the first polymeric layer (A) to cover ≤70%, preferably ≤50%, more preferably ≤30%, most preferably ≤ 20% of the first part of the first polymeric layer (A) in step (II) can be established in any manner the person skilled in the art would select for providing a barrier layer (D). Preferably, the providing of a further layer (D) is selected from the group consisting of screen printing, brushing, submitting a film or a combination of at least two thereof. Preferably, the providing in step (II) is performed by a screen printing process of a material with an MVTR of ≤500 g/m²d.

Optionally applying of at least one electrical pathway onto the first polymer layer (A) in step (III) can be established by any means the person skilled in the art would select for the process. The applying is preferably selected from the group consisting of printing, brushing, dabbing, coating with a doctor knife or a combination of at least two thereof. Preferably, the applying of at least one electrical pathway onto the first polymer layer (A) in step (III) is performed by a printing process.

The printing of the electrical pathways in step (III), for example in the form of conductor tracks, can be established in any manner the person skilled in the art would select for printing electrical pathways. Preferably, the printing of electrical pathways is established by a method selected from the group consisting of ink-jet printing, gravure printing, extrusion based 3D-printing, off-set printing, screen-printing or a combination of at least two thereof. Preferably, the electrical pathways provide a width of ≤2 mm, more preferably of ≤1 mm, even more preferably of ≤0.5 mm, most preferably of ≤0.1 mm.

The providing of electronic device (B) can be established by any means the person skilled in the art would select. Providing of the electronic device (B) on the first part of the first polymeric layer (A) in step (IV), preferably on the surface are ep2) is preferably established on the further layer (D) which is deposited on the surface of second surface area ep2) of the first polymeric layer (A). The providing in step (IV) may be performed manually or automatically. The providing in step (IV) is preferably selected from the group consisting of manual pick & place in the roll to roll process, an automatic pick & place in the roll to roll process via robotics, a manual placing on a belt, an automatic placing on the belt, a manual placing on a conveyer, an automatic placing on the conveyer, an automatic placing on the substrate, a manual placing on the substrate or a combination thereof. The substrate may be chosen from any surface like a desk or a table.

Optionally providing a second further layer (D) onto the electronic device (B) in step (V) can be established in any manner the person skilled in the art would select for providing such a water barrier layer (D). Preferably, the providing in optional step (V) is performed in the same manner as in step (II).

Optionally providing of the foam (F) in step (VI) can be established by any means the person skilled in the art would select for the process. The providing of the foam in step (VI) is preferably selected from the group consisting of manually or automatically placing of the foam (F) preferably next to or on the electronic device (B). Covering of the electronic device (B) in step (VII) can be established by any means the person skilled in the art would select for the process. The covering in step (VII) is preferably selected from the group consisting of manually or automatically covering of the electronic device (B) by the polymeric layer (A) or by a second polymeric layer (C). Preferably, the covering in step (VII) is established in the same manner as the providing of the first layer (A) in step (I). The automatic covering is preferably established via a roll to roll process. The second polymeric layer (C) preferably comprises the same materials in the same amount as the first polymeric layer (A).

Optionally fixing the overlapping edges of the first part of the first polymeric layer (A) and the second part of the first polymeric layer (A) or fixing the second polymeric layer (C) with the first part of the first polymeric layer (A) achieving an electronic patch (EP) in step (VIII) can be established by any means the person skilled in the art would select for fixing different films or foams. Preferably, the fixing in step (VIII) is selected from the group consisting of welding of the edges of the outer layers (A) or (A) and (C), laminating the whole sandwich of layers and devices, gluing at least the edges of the outer layers (A) or (A) and (C), clamping the edges of the outer layers (A) or (A) and (C) or a combination of at least two thereof. Preferably, the fixing in step (VIII) is performed by a lamination of the whole sandwich of layers and devices at temperatures of from 40° C. to 350° C., preferably from 60 to 250° C., more preferably from 70 to 220° C., most preferably from 80 to 200° C. or a welding at these temperatures of just the edges of the overlapping layers (A) and optionally layer (C).

Preferably, the dimensions and the orientation of the first polymeric layer (A), optionally of the second polymeric layer (C) and optionally of the further layer (D) are chosen in a way that the overall breathability of the electronic patch (EP) is in a range of 50 to 100% of the breathability of layer (A).

In a preferred embodiment of the process the fixing in step (VIII) is achieved by welding the polymeric layers (A) and optionally (C) at their overlapping edges, preferably at a temperature in a range of 40 to 350° C., preferably from 60 to 250° C., more preferably from 70 to 220° C., most preferably from 80 to 200° C.

Optionally providing an adhesive onto the underside of the electronic patch (EP) can be established by any means the person skilled in the art would select for providing an adhesive to a surface. Preferably, the providing of the adhesive is selected from the group consisting of a polyurethane adhesive, an acrylic adhesive, a silicone adhesive, a hydrogel adhesive or a combination or mixture of at least two thereof. Preferably, the adhesive is a biocompatible adhesive for application on the skin of a user. Preferably, the adhesive is a Baymedix® A of Covestro AG, Germany.

According to the invention at least two process alternatives exist to manufacture the electronic patch (EP). The first process alternative results in an electronic patch (EP) shown in FIG. 2. The second process alternative is illustrated in FIG. 3.

In the first process alternative the optional steps (II) and (III) are provided. Preferably, step (II) is established by a screen printing process of a water barrier material as described above. The dimension of the further layer (D) which is applied onto the first polymeric layer (A) is chosen in a way, that only the electronic device (B) can be placed on it in step (IV). Preferably, step (III) is established by an printing process to form electrical pathways as shown in FIG. 2. The electrical pathways are installed on the first polymeric layer (A) to establish a possibility to connect the electronic device (B) with an energy source or a further electrical device outside of the electronic patch (EP). If the electrical pathways are not protected by a water barrier before installation, the installation of the electrical pathways is preferably performed onto further layer (D). After deposition of the electronic device (B) onto the further layer (D) the electronic device (B) and if present also the electrical pathways will be covered by a second further layer (D) in step (V). In the case that the electronic device (B) is flat and all layers and devices deposited in steps (I) to (VII) will be laminated in step (VIII), a foam (F) is positioned on top of the electronic device or if present on top of the further layer (D) and at least the free spaces of the first polymeric layer (A). Also, in the case the fixing in step (VIII) will be performed solely at the edges of the first polymeric layer (A) and optionally the second polymeric layer (C), the foam (F) is preferably provided in step (VI).

All materials, like the first polymeric layer (A), the second polymeric layer (C), the electronic device (B), the further layer (D) and the foam (F) are selected from the respective materials already disclosed in connection with the electronic patch (EP) according to the invention.

The second process alternative is described in detail in the context of the description of FIG. 3.

A further aspect of the invention refers to an electronic patch (EP) obtained by a process according to the invention.

A further aspect of the invention refers to a use of an electronic patch (EP) according to the invention or produced by a process according to the invention for the application on the skin of a user.

### FIGURES

The FIGS. 1 to 3 show:

FIG. 1: a schematic view of a non-breathable arrangement of electronics, electrical pathways of a sensor of the prior art;

FIG. 2: a schematic view of an arrangement of electronics, electrical pathways of a sensor according to the invention;

FIG. 3: a scheme of process steps according to the process according to the invention.

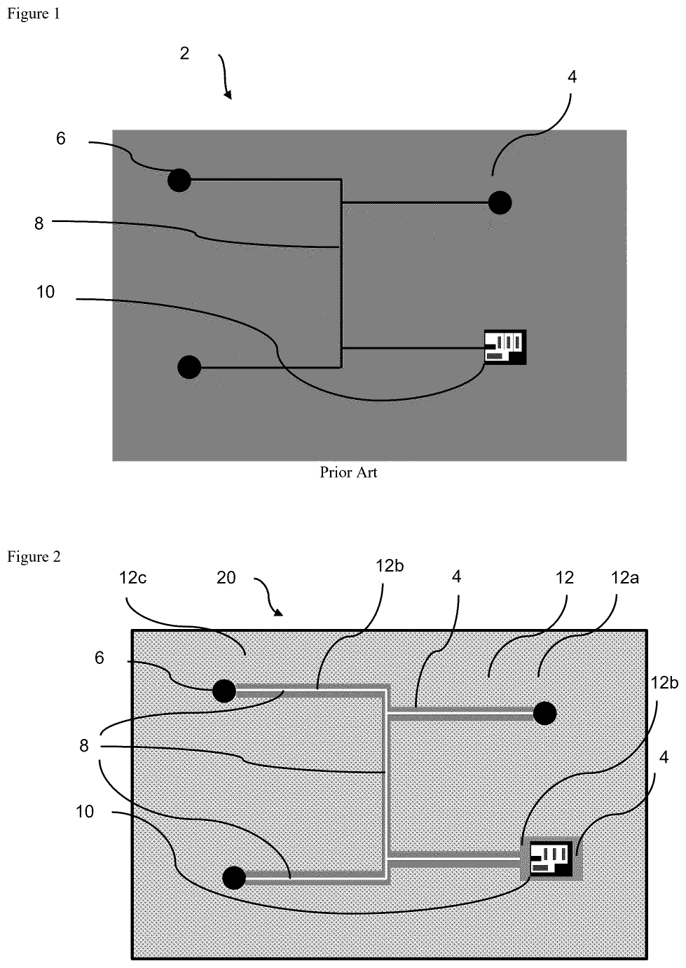

In FIG. 1 a scheme of a conventional electronic patch 2 is shown where a substrate (not shown) is covered by a water barrier 4 over the whole surface area of the substrate or the electronic patch. The electrical connections 6 and pathways 8 are positioned on the barrier 4 to connect the electronic device 10 with a further device (not shown).

In FIG. 2 a scheme of an electronic patch (EP) 20 according to the invention is shown. Here, a first polymeric layer (A) 12 in form of a water vapor-permeable TPU film, for example VPT 9101 T natural of Epurex Films GmbH & Co.KG is provided. The surface a) 12c of the first polymeric layer (A) 12 is divided in a first surface area ep1) 12a which is superimposed by material with an MVTR of ≥500 g/m²d and a second surface area ep2) 12b which is covered by material of the further layer (B) 4. The small areas of the second surface area ep2) 12b are covered by a water barrier material in form of further layer (D) 4. These areas 12b are screen printed by the water barrier material onto the first polymeric layer (A) 12. Onto these areas 12b of the first polymeric layer (A) 12 an electronic device 10 is deposited or printed and electrical pathways 8 are printed. The electrical pathways 8 connect the electronic device 10 with electrical connections 6. These electrical connections 6 can be used to connect the electronic device 10 with electronic devices, like batteries or computers in the surrounding of the electronic patch (EP). If needed, the electrical connection 6 might be physically connected through the overlaying layers (C), (A) and/or (D). In the example shown in FIG. 2, 85% of the surface a) 12c is covered by material with a moisture vapour transmission rate (MVTR) of ≥500 g/m²d and builds first surface area ep1) 12a. Accordingly, the second surface area ep2) 12b builds ≤15% of the total surface a) 12c.

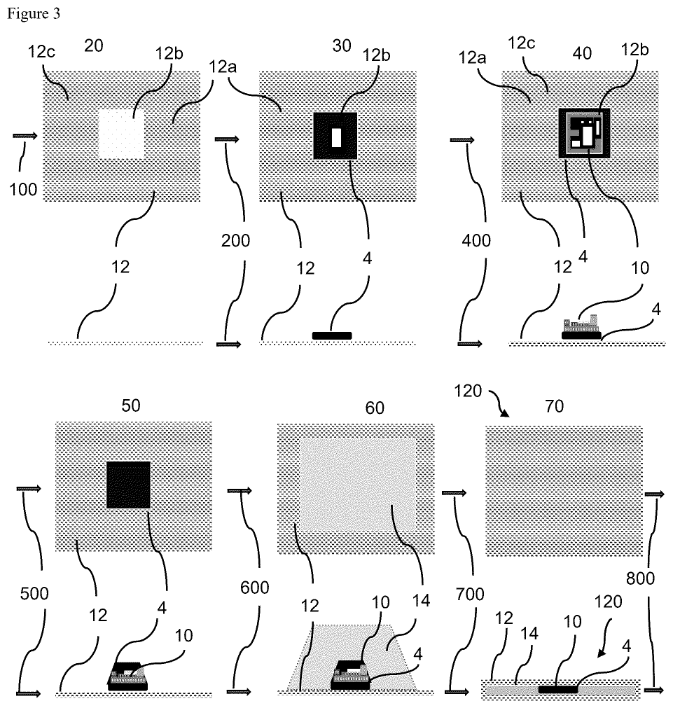

In FIG. 3, in the first scheme 20, a flat first part of a first polymeric layer (A) 12 comprising a first surface area ep1) 12a and a second surface area ep2) 12b, is shown. The upper view of the scheme 20 is a top view onto the first polymeric layer (A) 12 and the lower view of scheme 20 is a side view of the first polymeric layer (A) 12. The perspective of the views also applies to schemes 30, 40, 50, 60 and 70. According to the process according to the invention in step (I) 100 a water vapor-permeable TPU film in form of a VPT 9101 T natural of Epurex Films GmbH & Co KG a subsidiary of Covestro Deutschland AG, Germany in 25 μm thickness is provided.

In optional step (II) 200 a further layer (D) 4 may be provided in a conventional screen printing process onto the second surface area ep2) 12 of the first polymer layer (A) 12 providing surface a) 12c. As can be seen in the top view and also in the side view of scheme 30 the further layer (D) 4 is positioned in the middle of the first polymeric layer (A) 12. By applying this further layer (D) 4 a water vapor barrier is applied onto the first layer (A) 12. For this purpose the printing paste LOCTITE® EDAG PF 455B E&C from Henkel was applied by a normal screen printing process.

In step (IV) 400 an electronic device 10 is placed onto the further layer (D) 4 separating the first polymeric layer 12 from the electronic device 10. This is established by a manual placement (pick&place process).

In optional step (V) 500, a further water barrier layer (D) 4 may be applied onto the electronic device 10. This is established by applying printing paste LOCTITE® EDAG PF 455B E&C from Henkel by the screen printing process as described for step (II) or by a brushing process.

In optional step (VI) 600 a water vapor-permeable thermoplastic polyurethane foam (F) 14 from Mayser (Bluefoam MGT-160 in 1.5 mm thickness) may be applied onto a part of the first layer 12 as well as the surface of the electronic device 10. The foam (F) 14 may be provided directly on the electronic device 10 or onto the water barrier layer (D) 4 covering the electronic device 10 if applied in optional step (V).

In step (VII) 700 a further water vapor-permeable thermoplastic polyurethane film 12 is finally applied onto the structure built after step (VI) 600, especially onto the foam 14 which covers the electronic device 10 surrounded by the water barrier layer (D) 4. As material of the further film 12, a VPT 9101 T natural of Epurex Films GmbH & Co KG a subsidiary of Covestro Deutschland AG, Germany is applied in a thickness of 25 μm. This layer arrangement forms the electronic patch (EP) 120.

In a lamination process, in step (VIII) 800 the edges of the entire patch (EP) 120 are welded together at a temperature of 150° C. for 3 seconds.

Optionally, a breathable adhesive, for example Baymedix® A of Covestro AG, Germany is applied to the bottom of electronic patch (EP) 120, preferably directly onto the outer surface (ep) of the first polymeric layer 12.

In the example shown in FIG. 3, 80% of the surface a) 12c is covered by material with a moisture vapour transmission rate (MVTR) of ≥500 g/m²d and builds first surface area ep1) 12a. Accordingly, the second surface area ep2) 12b builds ≤20% of the total surface a) 12c.

### Methods

Moisture Vapour Transmission Rate (MVTR): all values for the moisture vapour transmission rate have been determined according to DIN 53122-1:2001-08.

1. An electronic patch (EP) providing at least one flat surface ep) which is arranged to be placed on the skin of a user, the electronic patch (EP) comprising at least:
(A) a first polymeric layer (A) with a first surface a) and a second surface b) which is parallel to surface a), whereby layer (A) provides a moisture vapor transmission rate (MVTR) of ≥500 g/m²d;
(B) an electronic device (B) providing at least one flat surface b) which is positioned on the first surface a) of the first polymeric layer (A), wherein the electronic device (B) is optionally surrounded by a water barrier (WB);
(C) optionally a second polymeric layer (C) providing a moisture vapor transmission rate (MVTR) of ≥500 g/m²d;
(D) optionally at least one further layer (D) as water barrier (WB) positioned between the electronic device (B) and the first polymeric layer (A) or between the electronic device (B) and the second polymeric layer (C) or both;
(E) optionally an adhesive (E) on the second surface b) of the first polymeric layer (A);
(F) optionally a polymeric foam (F) with a moisture vapor transmission rate (MVTR) of ≥500 g/m²d covering the electronic device (B);
wherein a surface area ep1) which covers ≥50% of the total surface of the flat surface ep) of the electronic patch (EP) is superimposed by material providing an overall moisture vapor transmission rate (MVTR) of ≥400 g/m²d.

2. The electronic patch (EP) according to claim 1, wherein ≥50%, of the total volume of the electronic patch (EP) is filled with a material that provides a moisture vapor transmission rate (MVTR) greater than 500 g/m²d.

3. The electronic patch (EP) according to claim 1, wherein the polymer of the first polymeric layer (A) and optionally the second polymeric layer (C) are each independently selected from the group consisting of a thermoplastic elastomer, a silicone, an (expanded) polytetrafluorethylene (PTFE), a polyamide, a polyphthalamide, a polyblockamide, a polyacrylate, a polycarbonate, a co-polycarbonate, a polyether-blockamide, a polyester, a co-polyester, a polyimide, a polyvinylchloride, a polyolefine and a combination of at least two of these.

4. The electronic patch (EP) according to claim 1, wherein the first polymeric layer (A) and optionally the second polymeric layer (C) provides at least one of the following properties:
(A1) a thickness in a range of 10 to 500 μm;
(A2) a surface area in a range of 1 to 1000 cm²;
(A3) an aspect ratio of thickness to length or width building the surface area is in a range of 1:10 to 1:10000;
(A4) vapor permeability of ≥500 g/m²d;
(A5) a vapor permeability in a range of 500 to 3000 g/m²d.

5. The electronic patch (EP) according to claim 1, wherein the electronic device (B) provides at least one of the following properties:
(B1) a thickness in a range of 0.5 μm to 5 cm;
(B2) a volume in a range of 0.5 to 500 mm³;
(B3) optionally, the electronic device is surrounded by a water barrier (WB);
(B4) at least one electronic component selected from the group consisting of antenna, battery, wires, rigid PCB boards, flexible PCB boards, chips, memory elements, sensors and combinations of at least two of these.

6. The electronic patch (EP) according to claim 1, wherein at least one of the at least one further layer (D) is present and is positioned between the electronic device (B) and the first polymeric layer (A), and optionally between the electronic device (B) and the second polymeric layer (C).

7. The electronic patch (EP) according to claim 1, wherein the ratio of the surface area of the first polymeric layer (A) to the surface area of the first layer (D) is in a range of from 100:1 to 1.5:1.

8. The electronic patch (EP) according to claim 1, wherein the further layer (D) is present and provides at least one of the following properties:
(D1) a thickness in a range of 1 to 500 μm;
(D2) a surface area in a range of 0.1 to 900 cm²;
(D3) a vapor permeability smaller than 400 g/m²d.

9. The electronic patch (EP) according to claim 1, wherein at least one of the at least one further layer (D) is present and is made of one selected from the group consisting of aluminum, a polymer foil, a foam, a foil/foam combination, a coated polymeric foil, a glass, a ceramic and a combination of at least two of these.

10. The electronic patch (EP) according to claim 1, wherein at least one of the at least one further layer (D) is present and is made of a polymer selected from the group consisting of polyethylene, polypropylene, polyethylene terephthalate, polystyrene, polyvinylchloride, polyamide, and a mixture of at least two of these.

11. The electronic patch (EP) according to claim 1, wherein the polymeric foam (F) is present and is selected from the group consisting of polyurethanes, thermoplastic polyurethanes, polyolefinic materials, silicones and a mixture or a combination of at least two of these.

12. A process for producing an electronic patch (EP) providing a flat surface ep) on its outside, the process comprising at least the following steps:
(I) providing a flat first part of a first polymeric layer (A) comprising a first surface area ep1) and a second surface area ep2), wherein the first polymeric layer (A) provides a vapor permeability in a range of 500 to 3000 g/m²d;
(II) optionally, providing a further layer (B) on the second surface area ep2) of the first part of the first polymeric layer (A) to cover ≤70% of the first part of the first polymeric layer (A);
(III) optionally applying of at least one electrical pathway onto first polymeric layer (A) and/or the further layer (D;
(IV) providing an electronic device (B) on the first part of the first polymeric layer (A) so that the electronic device (B) is not in direct contact with the polymeric layer (A);
(V) optionally, providing a second further layer (D) on the electronic device (B);
(VI) optionally providing a polymeric foam (F) with a vapor permeability in a range of 500 to 1000 g/m²d covering the electronic device (B);
(VII) covering the electronic device (B) by a second part of the first polymeric layer (A) or a second polymeric layer (C) so that overlapping edges of the first part of the first polymeric layer (A) and the second part of the first polymeric layer (A) or of the first part of the first polymeric layer (A) and the second polymeric layer (C) are formed;
(VIII) fixing the overlapping edges of the first part of the first polymeric layer (A) and the second part of the first polymeric layer (A) or fixing the second polymeric layer (C) with the first part of the first polymeric layer (A) achieving an electronic patch (EP) which is totally surrounded by the first polymeric layer (A) or partially surrounded by the first polymeric layer (A) and partially surrounded by the second polymeric layer (C);
(IX) optionally providing an adhesive onto the underside of the electronic patch (EP);
wherein the first surface area ep1) which covers ≥50% of the total surface of the flat surface ep) of the electronic patch (EP) is superimposed by material providing an overall moisture vapor transmission rate (MVTR) of ≥400 g/m²d.

13. The process according to claim 12, wherein the fixing in step (VIII) is achieved by welding the polymeric layers (A) and optionally (C) at their overlapping edges, preferably at a temperature in a range of 40° C. to 350° C.

14. An electronic patch (EP) obtained by the process according to claim 12.

15. (canceled)
